(19) United States
(12) Patent Application Publication
(10) Pub. No.: US 2014/0017721 A1
(43) Pub. Date: Jan. 16, 2014

(54) SPONTANEOUSLY IMMORTALIZED PROSTATE CANCER CELL LINE

(71) Applicant: Galina I. Botchkina, Stony Brook, NY (US)

(72) Inventor: Galina I. Botchkina, Stony Brook, NY (US)

(73) Assignee: The Research Foundation of State University of New York, Albany, NY (US)

(21) Appl. No.: 13/942,363

(22) Filed: Jul. 15, 2013

Related U.S. Application Data
(60) Provisional application No. 61/671,335, filed on Jul. 13, 2012.

(51) Int. Cl. G01N 33/50

(52) U.S. Cl. CPC G01N 33/5011
USPC 435/32; 435/325; 536/23.1; 530/350; 530/395

(57) ABSTRACT

This disclosure provides prostate cancer cell lines established from spontaneously immortalized, extremely tumorigenic and clonogenic primary prostate tumor. These cell lines represent unique cancer cell and cancer stem cell (CSC) models for preclinical prostate cancer studies and CSC-targeted drug development, which is of high value for pharmaceutic companies producing anti-cancer agents, as well as for the broad range of basic and translational research focused on cancer cell and CSC biology, stem cell behavior, cancer development and metastasis.

### CROSS-REFERENCE TO RELATED APPLICATIONS

This application claims priority to U.S. provisional application 61/671,335, filed Jul. 13, 2012, which is incorporated herein in its entirety.

### STATEMENT REGARDING FEDERALLY SPONSORED RESEARCH OR DEVELOPMENT

This invention was made with government support under Contract number 5R21CA150085-2 awarded by the National Institutes of Health/National Cancer Institute. The government has certain rights in the invention.

### BACKGROUND OF THE DISCLOSURE

Metastatic epithelial cancers presently have no known cure despite advances in screening and surgical treatment. Tumor regression induced by standard anti-cancer therapies does not correlate with patient survival, and the low effectiveness of standard therapies has been attributed to the existence of rare malignant cancer stem cells (CSCs) possessing tumor-initiating potential and maintaining tumor growth, spread, and resistance to treatment (Reya et al, 01; Clarke et al, 06). The existence of the CSCs is supported for the majority of human cancers, including prostate cancer (Collins et al, 05; Patrawala et al, 06; Miki et al, 07; Rowehl et al, 08; Klarmann et al, 09). The most alarming aspect of CSCs is their uninhibited proliferation in the presence of anti-cancer therapeutics. They are not only highly resistant to treatment, but usually quiescent CSCs are stimulated by conventional therapies to self-renew in order to repair and repopulate the damaged tumor with undifferrentiated drug resistant cells, thereby promoting cancer progression (Bao et al, 06; Dirks, 06; Eramo et al, 06; Todaro et al, 07; Bleau et al, 09; Tortoreto et al, 09).

It was recently demonstrated that treatment with 5-FU and oxaliplatin, a standard therapy for metastatic cancer, induced up to 30-fold enrichment of CSC expressing CD133+ and up to 2-fold enrichment of CD44+ cells (Dallas et al., 2009). Several major features of the CSCs make them very likely candidates to be the cause and driving force of metastasis, although metastatic progression depends on multiple factors (Shen & Abate-Shen, 11). Since CSCs are the only cell population with tumorigenic potential, it is conceivable that metastases-initiating cells should have CSC capabilities, and only multipotent CSCs have inherent plasticity to survive in a foreign environment and to propagate into a heterogeneous metastatic tumor. Recent data shows that CSCs can also differentiate into endothelial cells, thereby generating the necessary vasculature to fuel further tumorigenesis (Hutchinson, 11). Cells with metastatic activity were detected recently in multiple types of primary and metastatic tumors and metastatic cell lines (Mimeault & Batra, 10). Therefore, due to extreme clinical and biological significance of the CSCs, novel strategies must be developed for their targeted elimination or differentiation.

Prostate cancer (PrC) remains a major public health problem and the second leading cause of cancer deaths among men (Jemal et al., 2011), and the underlying mechanisms of the prostate carcinogenesis are poorly understood. Currently, a major obstacle in the field is the well known inability to grow freshly dissociated primary prostate tumor cells in vivo and in vitro (Hynes & Kelly, 2012). That is why cancer cell lines are largely used as a model system to examine the process of malignant transformation and to develop anti-cancer treatment strategies.

Historically, the majority of cancer-related studies are performed on the established cancer cell lines grown as a monolayer culture, which has little relevance to three-dimensional primary tumors, as well as no biological relevance to CSCs. In addition, there are only three readily available long-term human prostate carcinoma cell lines, DU-145, PC-3 and LNCaP, all of which were isolated from metastatic lesions, and as such, it is unlikely that these cell lines can accurately recapitulate the phenotypic, genomic and proteomic composition, as well as biological behavior of primary prostate tumors.

Long-term cell cultures can be established through the immortalization of primary tumor cells with transfection of the catalytic domain of the enzyme telomerase (hTERT), the E6 and E7 genes of the human papilloma virus 16, HPV-16, or the large T gene of the simian virus 40, SV40. However, these in vitro models are also not ideal, because some of the changes that occur are directly related to the activities of the particular oncogene used for transformation. Thus, the immortalized cells frequently contain viral oncogenic DNA and accompany major cytogenic alterations and growth deregulation, thereby introducing many genetic and epigenetic artifacts into these cells.

Cell lines more appropriate for cancer studies would be those derived from spontaneously immortalized cells isolated from primary tumors. Unfortunately, spontaneous immortalization is an extremely rare event, and there is a well-known difficulty in establishing long-term human epithelial cell lines, and especially primary prostate cancer cell lines, which has impeded efforts to understand prostate tumorigenesis and to develop alternative therapies for PrC. Even more rare is the identification and isolation of a spontaneously immortalized clone composed almost entirely of CSCs.

CSCs are not only functionally and morphologically different from cells making up the bulk of a tumor, but may themselves represent a heterogeneous phenotypic population. Although none of the currently available cell surface markers can be considered as universal or at least highly specific for CSCs, several methodological approaches have been developed allowing for reasonable purification and propagation of these cells.

Previous studies have identified the stem cell-related genome-wide (Rowehl et al., 2008) and proteome-wide characteristics of prostate CSCs and 3D spheroids induced by cells of a CD133^{high}/CD44^{high} phenotype isolated from the established highly metastatic prostate cancer cell line, PC3MM2. Genomic profiling with a stem cell-specific PCR array (SABiosciences) revealed that CD133^{high}/CD44^{high} prostate cancer cells, as well as the 3D spheroids induced by these cells, express profound up-regulation of the majority of the analyzed 84 stem cell-related genes in comparison to their differentiated counterparts. In addition, FACS and western blot analyses have shown that these cells contain some minority subpopulations with high levels of expression of several genes essential for pluripotency and self-renewal in embryonic stem cells, including Oct4, Sox2, Nanog and c-Myc.

CSCs represent the most critical target for anti-cancer therapeutic strategies, rational drug development and basic studies on cancer development and progression. In this context, a CSC-enriched cell line, which originates from spontaneously immortalized, highly tumorigenic and clonogenic primary prostate tumor, would have a high value for both pharmaceutical companies and basic research.

### BRIEF SUMMARY OF THE DISCLOSURE

This disclosure provides valuable cancer cell lines, methods of making such cell lines, and methods of use of such cell lines, for example in development of new cancer therapeutic drugs.

Although there is a complex regulation of the intrinsic dynamic equilibrium between CSCs and non-stem cancer cells, cancer cell lines provided by the disclosure can have up to 50-60% of total cells expressing CD44 and at least 4% of total cells expressing high levels of CD133. Cells from these cell lines are capable of anchorage-independent growth in culture, and are also capable of forming tumors in a xenograft model in immunodeficient animals, such as a mouse, rat, rabbit, dog, pig, or other model animal utilized in cancer studies. A preferred cell line with these characteristics is the PPT2 cell line.

The cancer cells of the invention express stem cell markers such as CD44, CD133, and CD326/epithelial cell adhesion molecule (EPCAM). A cell may express only one type of stem cell marker, or can express more than one stem cell marker. Preferred marker combinations include CD133^{high} and CD44^{high}, CD44^{high}/CD133^{high}, or CD44^{high}, CD133^{high}, and EPCAM^{high}.

This disclosure also provides a cancer stem cell (CSC) line that has at least 90% CD44+; EPCAM+ cells, and at least 50% CD133+; CD44+; EPCAM+ cells. Such a cell line also has cells capable of anchorage-independent growth in culture, and capable of forming tumors in a xenograft model. An example of a cell line with these characteristics is PPSS.

Methods of making the cancer cell lines described herein are also encompassed in this disclosure. Such methods include isolating tumor-derived cells that show fast adherence to type I collagen upon introduction to a tissue culture surface; performing one or more rounds of cell sorting; and culturing fast adherent cells under stem cell promoting conditions. Stem cell-promoting conditions include one or more of culturing cells in serum-free media; culturing cells on ultra-low-attachment culture surfaces; serial passage for two or more rounds on ultra-low-adherent culture surfaces; culturing at low cell density; and treatment under cytotoxic conditions. Additional steps to create these cell lines include serial transplantation in a xenograft model, and/or isolation and culturing of spheroid cells that show attachment-independent growth in culture.

This disclosure additionally presents methods of identifying candidate compounds with antiproliferative/anticancer activity using the cancer cell lines described herein. The methods include contacting cancer cells/CSCs from a cancer cell line described herein with a selected candidate compound; monitoring proliferation of the cancer cells/CSCs; identifying the candidate compound as an antiproliferative/anticancer/anti-CSC agent if the candidate compound inhibits proliferation of the cancer cell relative to proliferation of a cancer cell of the same cell type that is not contacted with the candidate compound.

This disclosure further encompasses pharmaceutical compositions that have as an active ingredient a compound identified as having antiproliferative activity according to the methods of the invention, or a derivative of such a compound.

Cancer cell lines, methods of creating such lines, and methods of identifying compounds for cancer treatment as encompassed by the invention include prostate, breast, and lung cancer cell lines and treatments. A preferred cancer is prostate cancer.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1C. Parental spontaneously immortalized prostate cancer cells isolated from primary tumor and grown under stemness-promoting conditions. (A) small population of cancer cells surrounded by fibrocytes; (B) significant increase in cell number after serial transplantations, cell sortings and growing under sternness-promoting conditions; (C) highly drug resistant large multinucleated cells.

FIGS. 2A-2D. Fast formation of NOD/SCID mice tumor xenografts (A-C) after transplantation of 1.5×10³ parental CD133+-enriched prostate cancer cells grown adherent to the type I collagen surfaces in serum-free stem cell medium. (A) tumor growth evident in mouse after transplantation; (B) tumor size; (C) graph of tumor take in comparison to the highly invasive PC3MM2 cells. (D) formation of 3D spheroids by cells grown under non-adherent culture conditions in serum-free medium.

FIGS. 3A-3B. Comparative expression of the stemness markers by primary parental and established highly invasive PC3MM2 prostate cell lines (A). Dose-dependent drug-induced increase in cells expressing stemness markers (B).

FIGS. 4A-4E. (A-B) PCR Array assay shows up-regulation of the majority of stemness genes (B) and transcription factors (A) in parental CD133⁺ cells. Western blot analysis (C) and immunocytochemistry (D) vimentin—red fluorescence; (E) nestin—green fluorescence); confirmed expression of multiple stem cell pluripotency markers. In addition, (C) shows the lack of pro-apoptotic proteins p53 and p21.

FIGS. 5A-5D. Formation of 3D spheroids by colonies of small immature cells. (A), subpopulation of small immature cells appearing as round colony (holoclone) surrounded by spindle-like much larger cells; (B-C), formation of 3D spheroids on cells adherent to type I collagen; (D), detached and floating multicellular spheroids.

FIGS. 6A-6D. (A-D) Formation of new colonies of small immature cells by plating adherent 3D spheroids on type I collagen-coated plates.

FIGS. 7A-7D. Phenotypic characteristics of the purified PPSS cells by FACS analysis. About 60% of cells express high levels of CD133 (A, B, D) compared to 5.3% in parental spheroid cells (C). The entire population is highly positive for CD44 and EPCAM. In contrast, only 3.3% of cells are differentiated (D), compared to 26.5% in the parental spheroid cells (C).

### DETAILED DESCRIPTION OF THE DISCLOSURE

This disclosure provides cell lines that contain high percentages of cancer stem cells (CSCs) derived from primary tumors. These cell lines have multiple characteristics of CSCs including anchorage-independent growth, ability to form tumors upon transplantation into a xenograft model organism, extensive proliferative capacity, and expression of stem cell markers such as CD133 and CD44. These cell lines further express pro-apoptotic genes such as p53 and p21 at reduced or non-existent levels.

Previously, the inventors have found that the prostate CD133^{high}/CD44^{high} cell phenotype (cells expressing CD133 and CD44 at high levels) isolated from several highly invasive prostate cancer cell lines retains high tumorigenic capacity during serial transplantations into immunodeficient NOD/SCID mice. Such cells also retained high clonogenic capacity during serial passaging of 3D floating spheroids in contrast to the majority of cancer cells which did not express these markers. In addition, these cells displayed characteristic stem cell plasticity under standard culturing conditions producing all the differentiated progeny. The inventors have now furthered these studies by creating methods of producing cancer stem cell-enriched cell lines, and have created highly valuable and rare cancer cell lines containing high levels of cancer stem cells.

Therefore, this disclosure provides cell lines with a greater percentage of cells expressing stem cell markers relative to previously known cancer cell lines. For example, a cell line according to the invention can have at least 2%, 3%, 4%, 6%, 10%, 20%, 30%, 40%, 50%, 60%, 70%, 80%, or 90% CD44+ cells. Further, a cell line according to the invention can have at least 2%, 3%, 4%, 6%, 10%, 20%, 30%, 40%, 50%, 60%, 70%, 80%, or 90% CD133+ cells. A cell line according to the invention can also have at least 2%, 3%, 4%, 6%, 10%, 20%, 30%, 40%, 50%, 60%, 70%, 80%, or 90% CD166+ cells. A cell line according to the invention can also at least 2%, 3%, 4%, 6%, 10%, 20%, 30%, 40%, 50%, 60%, 70%, 80%, or 90% CD326/epithelial cell adhesion molecule (EPCAM)+ cells. A cell line according to the invention can also have elevated percentages or numbers of cells, relative to other cancer cell lines, expressing combinations of stem cell markers, such as CD133+ and CD44+, or CD133+ and EPCAM+. Thus, a cell line according to the invention can have cells expressing, for example, at least 2%, 3%, 4%, 6%, 10%, 20%, 30%, 40%, 50%, 60%, 70%, 80%, or 90% CD133+; CD44+ cells (expressing both CD133+ and CD44+), or at least 10%, 20%, 30%, 40%, 50%, 60%, 70%, 80%, or 90% CD133+; EPCAM+ cells (expressing both CD133+ and EPCAM+), or at least 2%, 3%, 4%, 6%, 10%, 20%, 30%, 40%, 50%, 60%, 70%, 80%, or 90% CD133+; CD44+; EPCAM+ cells (expressing CD133+, CD44+, and EPCAM+).

In a particular example, this disclosure provides a primary cancer cell line, PPT2, established from spontaneously immortalized, extremely tumorigenic and clonogenic primary prostate tumor cells. The PPT2 line, and its derivatives, represent unique CSC models for preclinical prostate cancer studies and CSC-targeted drug development, which is of high value for pharmaceutical companies producing anti-cancer agents, as well as for the broad range of basic and translational research focused on the CSC biology, stem cell behavior, cancer development and metastasis. The PPT2 line is also a model cell line for prostate cancer studies, and additionally represents a source of further stem cell lines.

The PPT2 cell line is highly undifferentiated; that is, less than 10%, 7%, 5%, or 2% of the cell population expresses pan-keratin, a marker of cell differentiation. The PPT2 cell population is also at least 40%, 50%, 60%, 70%, 80%, 90%, or 95% CD133+, or at least 40%, 50%, 60%, 70%, 80%, or 90% CD133^{high}. The PPT2 cell population is also at least 80%, 85%, 90%, 93%, 95%, 97%, or 99% CD44+, or at least 80%, 85%, 90%, 93%, 95%, 97%, or 99% CD44^{high}.

As used herein, a “cancer stem cell” or “CSC” is defined according to the definition of cancer stem cell determined at the AACR Workshop (Clarke et al. Cancer Stem Cells—Perspectives on Current Status and Future Directions: AACR Workshop on Cancer Stem Cells. Cancer Res 66:9339, 2006) as: “a cell within a tumor that possesses the capacity to self-renew and to cause the heterogeneous lineages of cancer cells that comprise the tumor.” Cancer stem cells can thus be defined by their ability to recapitulate the generation of a continuously growing tumor. This definition encompasses the use of alternative terms in the literature, such as “tumor-initiating cell” and “tumorigenic cell” to describe putative cancer stem cells.

It must be emphasized that proliferation is not synonymous with self-renewal. A self-renewing cell division results in one or both daughter cells that have essentially the same ability to replicate and generate differentiated cell lineages as the parental cell. Stem cells have the ability to undergo a symmetrical self-renewing cell division, causing identical daughter stem cells that retain self-renewal capacity, or an asymmetrical self-renewing cell division, resulting in one stem cell and one more differentiated progenitor cell. In addition, it is thought that stem cells may divide symmetrically to form two progenitor cells, which could lead to stem cell depletion. Promoting this form of division is a way to deplete the cancer stem cell population and constitutes an alternative strategy for inducing cell death to treat cancer.

This disclosure provides valuable, spontaneously immortalized, primary prostate cancer cell lines with an enriched ratio of tumor-initiating cells (CSCs), specifically the cell line PPT2. This disclosure also provides the methods of maintenance and CSC enrichment in these rare, clinically relevant, ultra-low passage prostate cancer cell lines. The methods include maintenance of the PPT2 cells as subcutaneously-induced NOD/SCID mice tumor xenografts and 3D floating tumor spheroids; isolating cells from a xenograft tumor that show fast adherence to type I collagen in culture; culturing fast adherent cells under stem cell-promoting conditions; and performing one or more rounds of cell sorting to enrich the population of cells expressing stem cell markers. Such methods can also include the steps of at least one round of transplantation of cells in a xenograft model (preferably two or more serial rounds of xenotransplantation); and isolation and separate culturing of spheroid cells which show attachment-independent growth.

“Fast adherent” or “rapid adherent” cells refers to cells which show adherence to a tissue culture surface, for example a coated plate such as type I collagen-coated plate, within an hour of transfer onto the tissue culture surface, preferably adhering within 30 minutes, even more preferably adhering within 20 minutes.

“Ultra-low attachment” refers to culture conditions on a surface that minimizes cell attachment of cultured cells. One example of an ultra-low attachment surface is a culture vessel wherein the culture surface is coated with a covalently bound hydrogel layer that is hydrophilic and neutrally charged, such as CORNING™ brand ultra-low attachment (ULA) culture dishes and flasks.

As used herein, “stemness” refers to having characteristics of stem cells. “Tumorigenic” refers to ability or degree to which a cell can form tumors, such as by following transplantation in a xenograft model. “Clonogenic” refers to the ability or degree to which a cell can form clones (identical cells), which can be measured for example by determining plating efficiency (ability to form colonies) following cellular insult such as radiation or cytotoxic treatment. “Stemness-promoting” or “stem cell-promoting” conditions refer to conditions which increase the expression of stem cell characteristics in a population of cells. Such conditions include culturing CD133^{+/high} or CD133^{+/high}/CD44^{high} cells on ultra-low-adherent culture surfaces (such as Corning ULA tissue culture plates or flasks), serial passage (passage for two or more rounds) on type I collagen-coated surfaces, and preferential propagation of the cells which survive treatment under cytotoxic conditions such as treatment with cytotoxic compounds.

Cell sorting refers to separation of cells according to expression of cell surface antigens.

Non-limiting examples of cancers that can be utilized to create cancer cell lines in accordance with the invention include: leukemias; lymphomas; multiple myelomas; bone and connective tissue sarcomas; brain tumors; breast cancer; adrenal cancer; thyroid cancer; pancreatic cancer; pituitary cancers; eye cancers; vaginal cancers; cervical cancers; uterine cancers; ovarian cancers; esophageal cancers; stomach cancers; colon cancers; rectal cancers; liver cancers; gallbladder cancers; cholangiocarcinomas; lung cancers; testicular cancers; prostate cancers; penile cancers; oral cancers; basal cancers; salivary gland cancers; pharynx cancers; skin cancers; kidney cancers; Wilms' tumor; bladder cancers. In one example, the cancer is prostate, breast, colon, pancreatic, lung, gastric, or bladder cancer.

### Use of Cancer Cell Lines in Discovery of Cancer Treatments

The rare prostate cancer cell lines disclosed herein are clinically relevant in relation to primary prostate tumors, and physiologically relevant in relation to stem cells. These cell lines will, for example, allow identification and screening of candidate compounds that are able to either directly kill tumor-initiating cells, suppress their stemness, or promote their differentiation into non-stem cells. The methods of screening candidate compounds involve contacting a cancer stem cell from the cell lines described herein with a candidate compound and monitoring proliferation of the cancer stem cell. A candidate CSC-targeting compound is identified as an antiproliferative or pro-apoptotic agent if the candidate compound inhibits proliferation or kills tumor-initiating CSCs compared to cancer cells from the same cell line that were not contacted with the candidate compound.

Monitoring of surviving cancer cells following treatment with a candidate agent, and evaluation of molecular alterations associated with their stemness state, can provide improved identification of CSC-targeting anti-cancer agents. The monitoring can be for one or more days, one or more weeks, or one or more months after treatment with the candidate agent. Such monitoring can include determining expression of stem cell-relevant cell surface markers (including, but not limited to CD133, CD44 and EpCAM), expression of pluripotentcy and stemness-related genes and proteins, and expression of pro-apoptotic and anti-apoptotic genes and proteins in the surviving cancer cells. Ability to suppress expression of stem cell-relevant markers, suppress expression of pluripotentcy and stemness-related genes and proteins, suppress anti-apoptotic genes, and/or up-regulate pro-apoptotic genes, suggest a candidate compound is potentially efficacious as an anti-cancer drug.

As used herein, a “test compound” or “candidate compound” can be any chemical compound, for example, a macromolecule (e.g., a polypeptide, a protein complex, glycoprotein, or a nucleic acid) or a small molecule (e.g., an amino acid, a nucleotide, an organic or inorganic compound). A test compound can have a formula weight of less than about 10,000 grams per mole, less than 5,000 grams per mole, less than 1,000 grams per mole, or less than about 500 grams per mole. The test compound can be naturally occurring (e.g., an herb or a natural product), synthetic, or can include both natural and synthetic components. Examples of test compounds include antioxidants, compounds that structurally resemble antioxidants, peptides, peptidomimetics (e.g., peptoids), amino acids, amino acid analogs, polynucleotides, polynucleotide analogs, nucleotides, nucleotide analogs, and organic or inorganic compounds (e.g., heteroorganic or organometallic compounds).

Test compounds can be screened individually or in parallel. An example of parallel screening is a high throughput drug screen of large libraries of chemicals. Such libraries of test compounds can be generated or purchased, e.g., from Chembridge Corp., San Diego, Calif. Libraries can be designed to cover a diverse range of compounds. For example, a library can include 500, 1000, 10,000, 50,000, or 100,000 or more unique compounds. Alternatively, prior experimentation and anecdotal evidence can suggest a class or category of compounds of enhanced potential. A library can be designed and synthesized to cover such a class of chemicals.

The synthesis of combinatorial libraries is well known in the art and has been reviewed (see, e.g., Gordon et al., J. Med. Chem., 37:1385-1401, (1994); DeWitt, and Czarnik, Acc. Chem. Res., 29:114, (1996); Armstrong, et al., Acc. Chem. Res., 29:123, (1996); Ellman, J. A. Acc. Chem. Res., 29:132, (1996); Gordon, et al., Acc. Chem. Res., 29:144 (1996); Lowe, G. Chem. Soc. Rev., 309 (1995); Blondelle et al. Trends Anal. Chem., 14:83 (1995); Chen, et al., J. Am. Chem. Soc., 116:2661 (1994); U.S. Pat. Nos. 5,359,115, 5,362,899, and 5,288,514; and PCT Publication Nos. WO92/10092, WO93/09668, WO91/07087, WO93/20242, and WO94/08051).

Libraries of compounds can be prepared according to a variety of methods, some of which are known in the art. For example, a “split-pool” strategy can be implemented in the following way: beads of a functionalized polymeric support are placed in a plurality of reaction vessels; a variety of polymeric supports suitable for solid-phase peptide synthesis are known, and some are commercially available (for examples, see, e.g., M. Bodansky, Principles of Peptide Synthesis, 2nd ed., Springer-Verlag, Berlin (1993)). To each aliquot of beads is added a solution of a different activated amino acid, and the reactions are allowed to proceed to yield a plurality of immobilized amino acids, one in each reaction vessel. The aliquots of derivatized beads are then washed, “pooled” (i.e., recombined), and the pool of beads is again divided, with each aliquot being placed in a separate reaction vessel. Another activated amino acid is then added to each aliquot of beads. The cycle of synthesis is repeated until a desired peptide length is obtained. The amino acid residues added at each synthesis cycle can be randomly selected; alternatively, amino acids can be selected to provide a “biased” library, e.g., a library in which certain portions of the inhibitor are selected non-randomly, e.g., to provide an inhibitor having known structural similarity or homology to a known peptide capable of interacting with an antibody, e.g., the an anti-idiotypic antibody antigen binding site. It will be appreciated that a wide variety of peptidic, peptidomimetic, or non-peptidic compounds can be readily generated in this way.

The “split-pool” strategy can result in a library of peptides, e.g., modulators, which can be used to prepare a library of test compounds of the invention. In another illustrative synthesis, a “diversomer library” is created by the method of Hobbs DeWitt et al. (Proc. Natl. Acad. Sci. USA, 90:6909 (1993)). Other synthesis methods, including the “tea-bag” technique of Houghten (see, e.g., Houghten et al., Nature, 354:84-86 (1991)) can also be used to synthesize libraries of compounds according to the subject invention.

Libraries of compounds can be screened to determine whether any members of the library have a desired activity and, if so, to identify the active species. Methods of screening combinatorial libraries have been described (see, e.g., Gordon et al., J. Med. Chem., supra). After screening, compounds that have a desired activity can be identified by any number of techniques (e.g., mass spectrometry (MS), nuclear magnetic resonance (NMR), matrix-assisted laser desorption ionisation/time of flight (MALDI-TOF) analysis, and the like). Exemplary assays useful for screening libraries of test compounds are described herein.

### Cell-Based Proliferation Assays

Cell-based proliferation assays can be used to assess the antiproliferative activity of a candidate compound against CSCs and bulk tumor cells. Generally, the assays include contacting a candidate compound to cancer cells of the cell lines described herein, such as PPT2 or PPSS cell lines, and subsequently assaying the effect of the candidate compound on proliferation of the cancer stem cells. A candidate compound is a potential antiproliferative CSC-targeting agent if the compound reduces proliferation of cancer stem cells, preferably in addition to reducing proliferation of bulk tumor cells, relative to similar cells that are not exposed to the candidate compound.

A number of proliferation assays are based on the incorporation of labeled nucleotide or nucleotide analogs into the DNA of proliferating cells. In these assays cells are exposed to a candidate compound and to a labeled nucleotide, e.g., .sup.14C-thymidine, .sup.3H-thymidine, or 5-bromo-2-deoxyuridine (BrdU). Proliferation is quantified by measuring the amount of labeled nucleotide taken up by the cells. Radiolabeled nucleotides can be measured by radiodetection methods; antibodies can be used to detect incorporation of BrdU. Other assays rely on the conversion of chemical precursors to a dye in dividing cells. Some assays measure the conversions of tetrazolium salts (e.g., methyl thiazole tetrazolium (MTT), 2-(4-Iodophenyl)-3-(4-nitrophenyl)-5-(2,4-disulfophenyl)-2H-tetraz-olium (WST-1), or 3′-{1-[(phenylamino)-carbonyl]-3,4-tetrazolium}-bis(4-methoxy-6-nitro) benzene-sulfonic acid hydrate (XTT)) to formazan by cellular mitochondrial dehydrogenases. Mitochondrial dehydrogenase activity increases in proliferating cells, thereby increasing the amount of formazan dye. The amount of formazan dye measured by absorbance is an indication of proliferation. Preferably, the MTT and other assays for evaluation of CSC-targeted antiproliferative efficacy is carried out on purified CSC populations grown on type I collagen-coated surfaces, to maintain the stemness state (Kirkland et al., 2009) of the tested CSCs in vitro and during treatment.

Still other assays measure cellular proliferation as a function of ATP production. For example, the luciferase enzyme catalyzes a bioluminescent reaction using the substrate luciferin. The amount of bioluminescence produced by a sample of cells measures the amount of ATP present in the sample, which is an indicator of the number of cells. Some cell proliferation assays directly measure the number of cells produced by a number of founder cells in the presence of a candidate compound. For example, in soft-agar colony formation assays, the cancer cells are suspended in agar-containing nutrient containing medium. Cells are incubated under conditions that allow for cell proliferation in the absence of a candidate compound. Colonies that form, if any, are stained with dye, e.g., crystal violet, and counted.

A further CSC-relevant assay for evaluation of the anti-cancer drug efficacy is an ability to form secondary floating spheroids. This sphere-forming assay is based on induction of the 3D spheroids by known number of cancer cells with particular phenotype (for example, CD133^{+/high} or CD133^{+/high}/CD44^{high} cells versus CD133- and CD44-negative cells), and then comparative evaluation of the sphere-forming capacity of the control untreated versus drug treated spheroids. Currently, this model is recognized as both clinically and biologically relevant (Friedrich et al., 2009).

### In Vivo Proliferation Assays

Candidate compounds can also be further tested for the ability to prevent proliferation of cancer cells in vivo. For example, the assays can involve administering a candidate compound to a xenograft animal model. In these assays, a known number of cancer cells from the cell lines described herein are transplanted into animals, e.g., immune-deficient NOD/SCID mice. One or more test animals are treated with a pharmaceutical composition that includes a candidate compound. One or more control animals are treated with a pharmaceutical composition lacking the candidate compound. The proliferation of cancer tissues (e.g., by measuring tumor size, tumor volume, and/or tumor weight) in the two sets of animals is assessed. If a candidate compound reduces the amount of cancer cell proliferation in one or more test animals, relative to control animals, then this candidate compound can be considered as an anti-cancer (or tumor debulking/tumor shrinking) agent.

In order to determine whether or not this agent possesses any anti-CSC efficacy, residual tumors can be analyzed for the presence of CSCs. Cells which survived treatment with the candidate agent can be analyzed for molecular alterations induced by this agent, including but not limited to the expression of the common cell surface markers of stemness, such as CD133, CD44 and EpCAM, expression of the pluripotency and stemness-relevant genes and proteins, and expression of pro-apoptotic and anti-apoptotic genes and proteins. Ability to suppress expression of common stem cell surface markers, suppress expression of pluripotentcy and stemness-related genes and proteins, suppress anti-apoptotic genes, and/or up-regulate pro-apoptotic genes, suggest a candidate compound is potentially efficacious as an anti-cancer drug.

### Medicinal Chemistry

Once candidate compounds, including candidate compounds that are antioxidant agents and/or antiproliferative agents have been identified, the compounds can be formulated for the treatment of diseases associated with ROS, e.g., cancer, or standard principles of medicinal chemistry can be used to produce derivatives of the compound for the treatment of cancer.

A candidate compound that has positive in vivo results, such as inhibition of the stemness state, or decrease of the number of CSCs, or promotion of differention is a candidate therapeutic agent. Candidate therapeutic agents can be optimized, derivatized, or made into pharmaceutical composition for clinical trials. Candidate therapeutic agents effective in clinical trials are therapeutic agents for treatment of cancer.

Derivatives can be screened for improved pharmacological properties, for example, efficacy, pharmaco-kinetics, stability, solubility, and clearance. The moieties responsible for a compound's activity in the assays described above can be delineated by examination of structure-activity relationships (SAR) as is commonly practiced in the art. A person of ordinary skill in pharmaceutical chemistry could modify moieties on a candidate compound or agent and measure the effects of the modification on the efficacy of the compound or agent to thereby produce derivatives with increased potency. For an example, see Nagarajan et al., J. Antibiot., 41:1430-8 (1988). Furthermore, if the biochemical target of the compound (or agent) is known or determined, the structure of the target and the compound can inform the design and optimization of derivatives. Molecular modeling software is commercially available (e.g., from Molecular Simulations, Inc.) for this purpose.

### Pharmaceutical Compositions

Candidate compounds, and/or derivatives thereof, can be incorporated into pharmaceutical compositions. Pharmaceutical compositions typically include a candidate compound, or a derivative thereof, and a pharmaceutically acceptable carrier. A “pharmaceutically acceptable carrier” can include solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (e.g., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injection include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR™ EL (BASF, Parsippany, N.J.) or phosphate buffered saline (PBS). In all cases, the composition should be sterile and fluid to the extent that easy syringability exists. It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be achieved by including an agent which delays absorption, e.g., aluminum monostearate or gelatin, in the composition.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Pharmaceutically compatible binding agents, and/or adjuvant materials can be included as part of the composition. The tablets, pills, capsules, troches, and the like can contain any of the following ingredients or compounds of a similar nature: a binder such as microcrystalline cellulose, gum tragacanth, or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the active compound(s) are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compound(s) can also be prepared in the form of suppositories (e.g., with conventional suppository bases such as cocoa butter and other glycerides) or retention enemas for rectal delivery.

In one embodiment, the active compound(s) are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue, e.g., bone or cartilage, in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The skilled artisan will appreciate that certain factors influence the dosage and timing required to effectively treat a patient, including but not limited to the type of patient to be treated, the severity of the disease or disorder, previous treatments, the general health and/or age of the patient, and other diseases present. Moreover, treatment of a patient with a therapeutically effective amount of an active compound can include a single treatment (e.g., for imaging) or, preferably, can include a series of treatments. Appropriate doses of the compound depend upon the potency of the small molecule with respect to the expression or activity to be modulated. When one or more of these small molecules is to be administered to an animal (e.g., a human) to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The pharmaceutical compositions can be included in a container, pack, or dispenser together with instructions for administration. For example, pharmaceutical composition that includes one or more compound of interest can be packaged together with a pharmaceutical composition that includes an antioxidant. Such packaging makes administration of the combination therapies disclosed herein.

The invention is further described in the following examples, which do not limit the scope of the invention described in the claims.

### EXAMPLES

### Example 1

### Establishment and Characterization of the Primary Prostate Cancer Cell Line PPT2

Needle biopsies were taken from otherwise discarded surgical waste (removed prostate glands) from operations already scheduled by their physicians for clinical care, in accordance with National Institute of Health guidelines. The prostate gland from which the parental cells were isolated was removed from a stage pT2c pNX pMX prostate cancer patient as a part of routine care for prostate cancer. Pathological staging: pT2—tumor invades beyond the organ or tissue or origin; pT2c—tumor affects both lobes; pNX—regional lymph nodes cannot be assessed; pMX—presence of distant metastasis cannot be assessed.

Needle biopsies were immediately digested with a cocktail of collagenases and antibiotics. First, tumor biopsies were minced with scissors into approximately 2 mm fragments (all procedures were carried out at sterile conditions), rinsed with Hank's balanced salt solution (HBSS) and incubated for 2 hours at 37° C. in serum-free RPMI medium 1640 containing 200 units/ml Collagenases type II and type IV (Sigma-Aldrich), 120 μg/ml penicillin and 100 μg/ml streptomycin. Cells were further disaggregated by pipetting and serial filtration through cell dissociation sieves (size 40 and 80 meshes; Sigma-Aldrich). Contaminating erythrocytes were lysed by incubation in ammonium chloride hypotonic buffer for 5 min on ice. Single cell suspensions were either used for further analyses immediately, or kept in liquid nitrogen in aliquots in freezing medium. Single cell suspension was plated on type I collagen-coated dishes in stem cell medium. Cells which adhered within 15-20 min (“fast adherent cells”) were collected and placed on ultra low-adherent plates or flasks to induce floating 3D spheroids. Alternatively, fast adherent cells remained on the type I collagen-coated dishes for further propagation. Propagated cells were then injected subcutaneously into 6-8 weeks old male NOD/SCID mice to monitor tumorigenesis and further propagate human cancer cells/CSCs.

Cells from only one out of 22 patient-derived specimens were able to survive in stringent conditions used for enrichment of cancer stem cells (CSCs). This spontaneously immortalized primary prostate tumor cell line has maintained extremely high tumor-initiating and sphere-forming capacities for greater than one year.

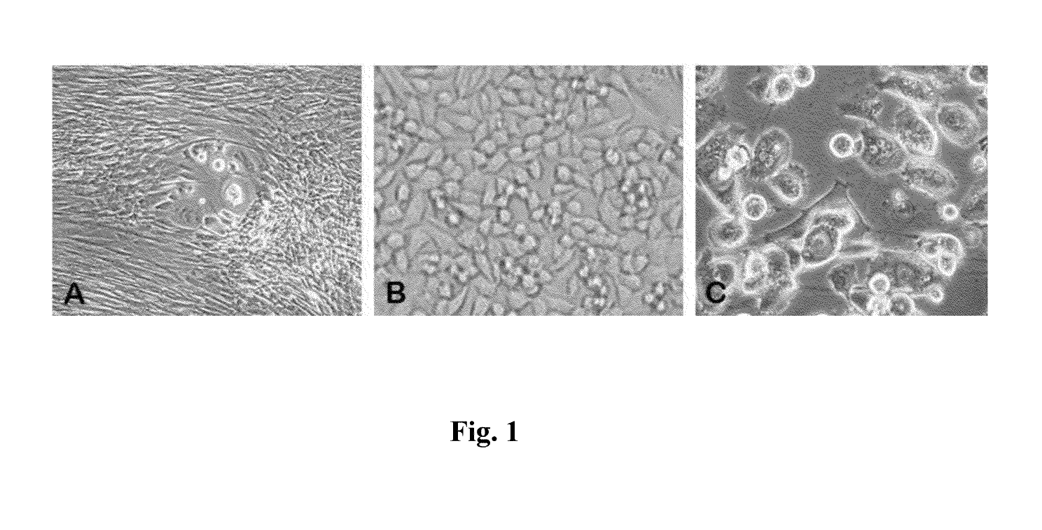

Initially, the isolated fast adherent cells represented a mixture of a majority of tumor-associated fibrocytes with a minor population of large cancer cells (FIG. 1A). After consequent cell sorting and culturing under sternness-promoting conditions, which include culturing on type I collagen-coated surfaces; culturing at non-adherent conditions (for example, at ultra-low-adherent ULA flasks and plates; Corning); culturing in serum-free stem cell-relevant media, such as mesenchymal stem cell media (Lonza); culturing at low cell density; and culturing in media containing 1% knock-out serum replacement (Invitrogen/Gibco), the population of prostate cancer cells—and more importantly, CSCs—was gradually increased (FIG. 1B). Treatment with cytotoxic drugs, including Taxol and SBT-1214 at 0.1-3 μM concentrations led to significant enrichment and visualization of the multinucleated gigantic cells, which represent a highly drug resistant type of CSCs (FIG. 1C).

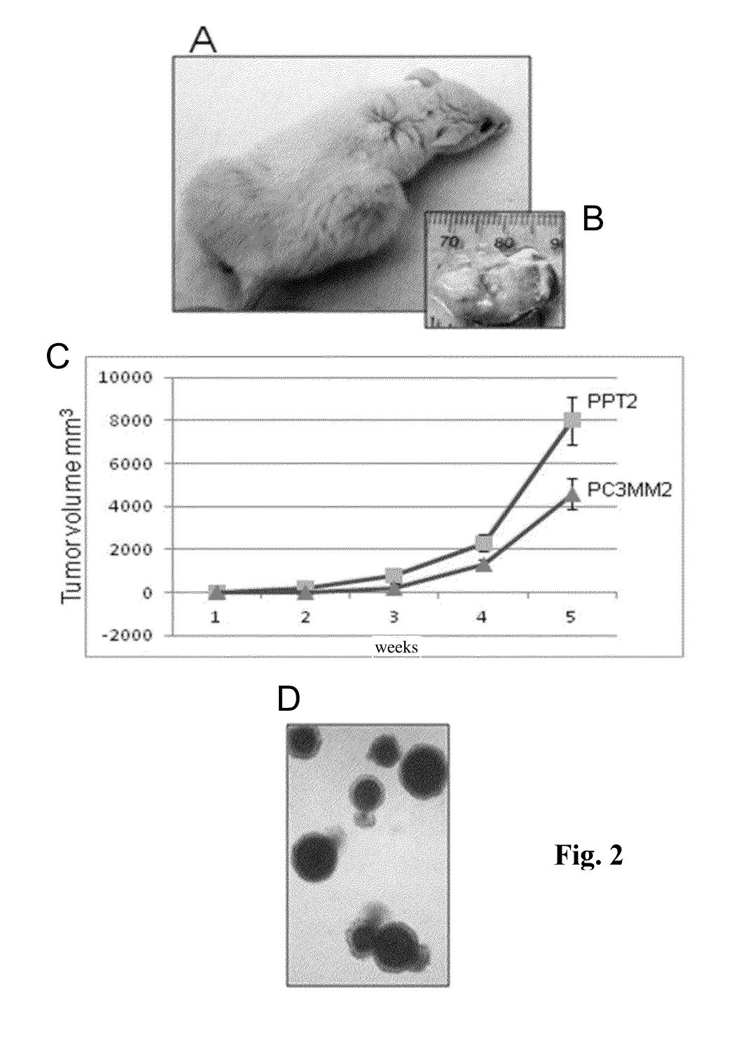

These parental cells were tested functionally using the two gold standard criteria of stemness: a) ability to induce tumors in immunodeficient mice; and b) ability to form anchorage-independent 3D cancer spheroids after serial transplantation of a low cell number in ULA flasks or plates. These cells possess unusually high tumor-initiating potential after transplantation into NOD/SCID mice of a low (1-3,000) cell number. Thus, these cells induced palpable tumors in 10 days, forming very large and vascularized tumors by 4-5 weeks (FIGS. 2A, 2B). For comparison, the same number of cells from the metastatic prostate CD133^{high} PC3MM2 cell line did not show palpable tumor formation before 21 days (FIG. 2C). In addition, these newly-established parental cells have high efficiency in forming 3D floating spheroids (FIG. 2D).

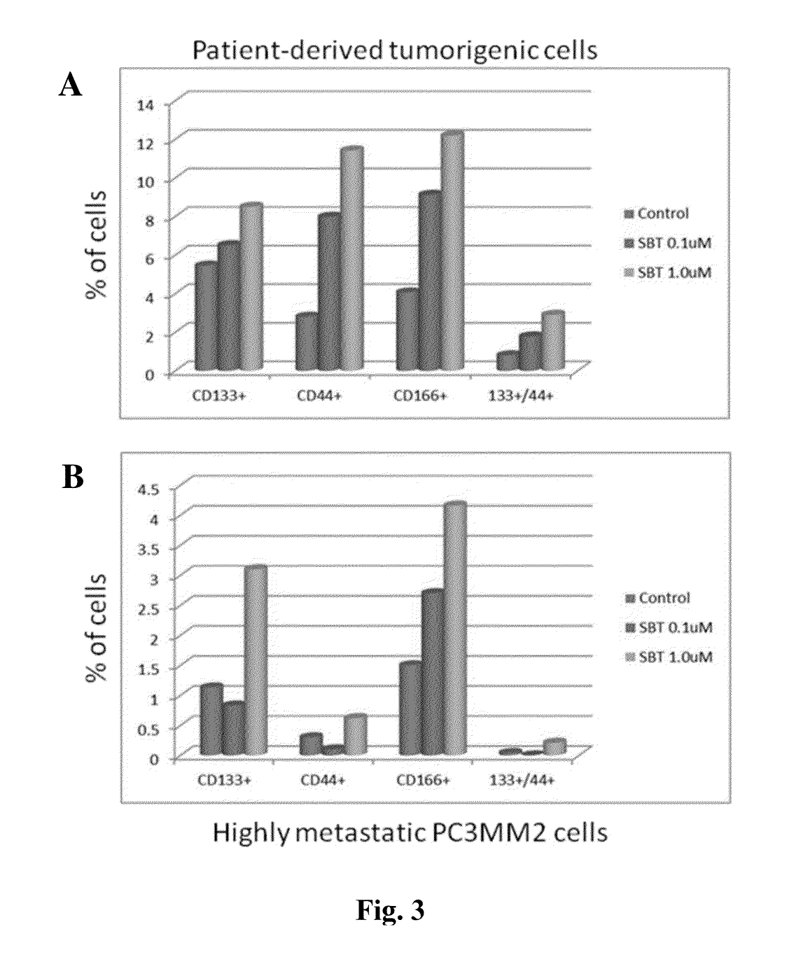

The parental cells contain relatively high ratios of known stem cell surface markers, including CD133 (2-11%), CD44 (50-90%), CD166 (up to 99%), combined expression of the CD133^{high} and CD44^{high} (FIG. 3A) and others, even in comparison to the aggressive PC3MM2 cell line.

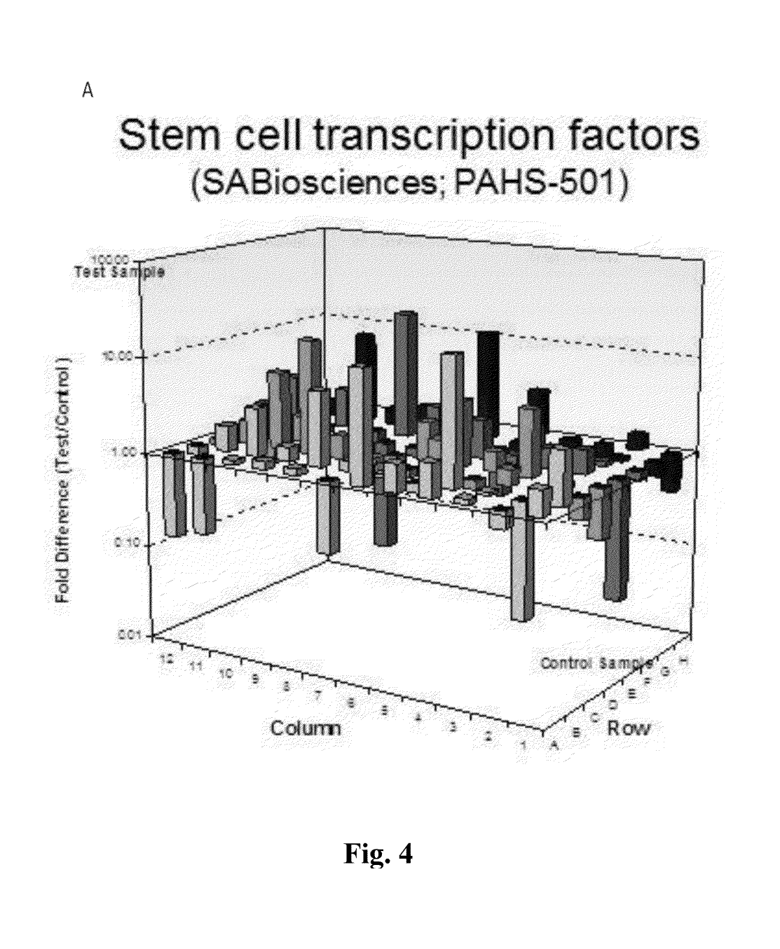

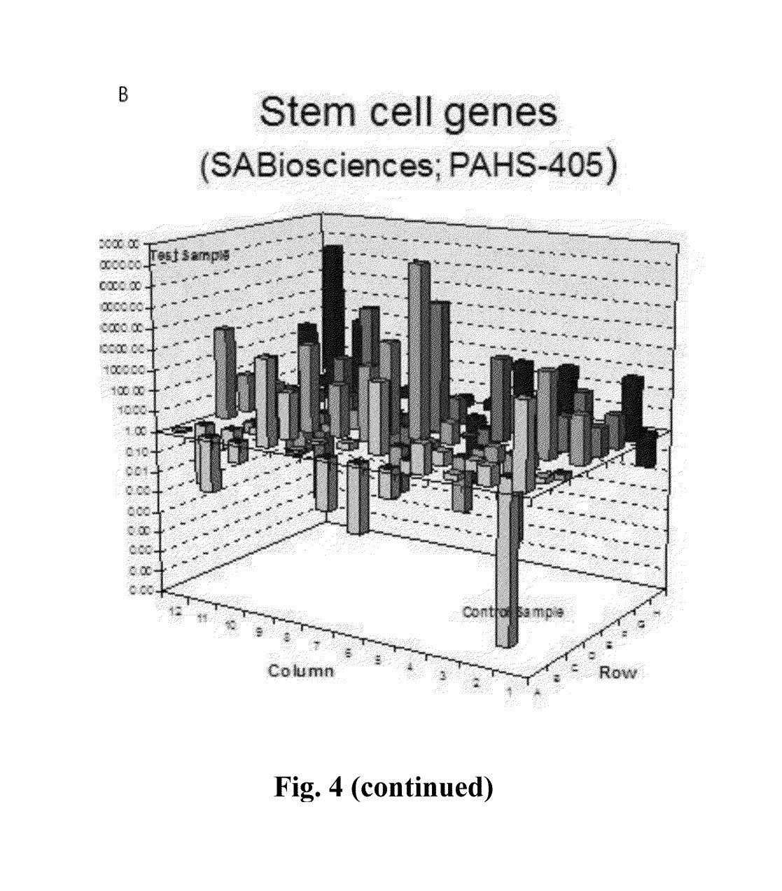

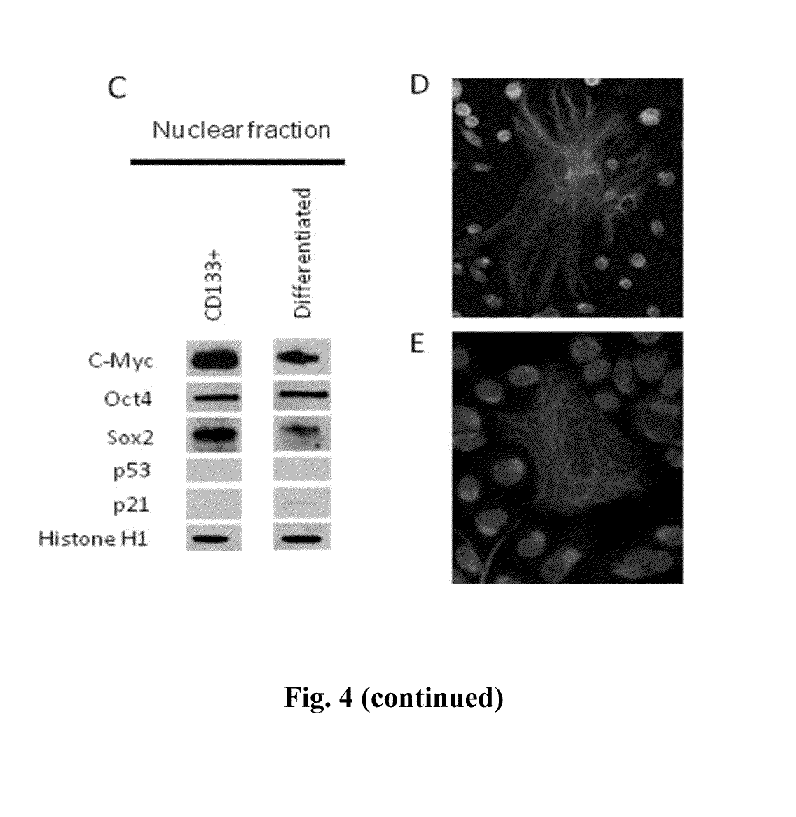

PCR Array analysis revealed that these cells possess up-regulated levels of the majority of studied stemness genes and transcription factors (FIG. 4A-B). The most up-regulated genes in prostate CSCs versus differentiated cells (total 41 of 84=45% genes) were EGR, FOXP3, GLI2, HOXA2, HOXA7, HOXC10, HOXC6, IRX4, JUN, KLF2, NFATC1, NR2F2, PCNA, PITX3, POU4F1, SIX2, SOX2, TERT and WT1, as well as other significantly up-regulated genes, including CDX2, DLX2, DNMT3B, 3EZH2, FOXP3, HOXA10, HOXA11, HOXA3, HOXA7, HOXB3, HOXB8, HOXB5, HOXC9, HOXC4, HOXC5, ISL1, NKX2-2, PAX9, PITX2, POU5F1, RUNX1, SOX9, VDR and WRN. Among them, several key pluripotency transcription factors characteristic for embryonic stem cells, including c-Myc, Oct3/4 and Sox2, were identified. These cells strongly expressed other markers of pluripotent cells, including vimentin (FIG. 4D) and nestin (FIG. 4E). In addition, the parental cell line did not express pro-apoptotic proteins p53 and p21, which reflects the high cell resistance to cytotoxic treatment (FIG. 4C).

To ensure more reliable isolation of CSCs, cells were labeled with one or several markers conjugated with different fluorescent dyes, including anti-human CD133/2-APC (clone 293C3; Miltenyi Biotec, CA, USA); CD166-PE (clone 105902; R&D Systems, MN, USA); CD44-FITC (clone F10-44-2), CD44-PE (clone F10-44-2; Invitrogen/Biosources, USA); CD44v6-FITC (clone 2F10; R&D Systems, USA), EpCAM-FITC (Biosource, CA, USA), Pan-Keratin (C11)-Alexa Fluor® 488 (Cell Signaling) and all the isotype controls (Chemicon). Antibodies were diluted in buffer containing 5% BSA, 1 mM EDTA and 15-20% blocking reagent (Miltenyi Biotec) to inhibit unspecific binding to non-target cells. After 15 min incubation at 4° C., stained cells were sorted and analyzed with multiparametric flow cytometer BD FACSAria (Becton Dickinson, CA). Alternatively, dissociated cells were centrifuged at 950 g for 5 min at 4° C., rinsed with sterile MACS buffer (Miltenyi Biotec, CA) and labeled with CD133 Abs directly or indirectly conjugated with ferromagnetic beads (Miltenyi Biotec, CA) as recommended by manufacturer.

Currently, the tumorigenic and clonogenic PPT2 cell line containing a high ratio of CSCs is maintained and propagated as serial NOD/SCID mice tumor xenografts, floating 3D cancer spheroids and type I collagen-adherent cultures induced by a purified subpopulation of cells with high expression of CD133. Additional characteristics of the PPT2 cell line, compared with the unrelated prostate cancer cell line PC3MM2 are found in Table 1.

TABLE 1
Phenotypic profiling of PPT2 and PC3MM2
cells with FACS analysis*

| | | PPT2 cells** | | PC3MM2 cells*** | |
|---|---|---|---|---|---|
| | | | % of | | % of |
| | | Levels of | total | Levels of | total |
| Marker | Source | expression | cell # | expression | cell # |
| CD133 | Miltenyi | +++ | 75 ± 15 | ++/+++ | 3 ± 1 |
| | Bio | | | | |
| CD44 | Invitrogen | +++ | 99.5 ± 0.5 | ++/+++ | 7.5 ± 2.5 |
| | Clone # | | | | |
| | MEM-85 | | | | |
| CD44v6 | R&D Clone | +++ | 56 ± 16 | | |
| | # 2F10 | | | | |
| CD49f | BioLegends | +++ | 99.5 ± 0.5 | +++ | 94 ± 2 |
| CD166 | BD Biosci. | +++ | 99.3 ± 0.5 | ++/+++ | 86 ± 8 |
| EpCAM | Miltenyi Bio | +++ | 98 ± 0.5 | ++/+++ | 83 ± 5 |
| Pan-Kerat | Cell Signal. | ++ | 4 ± 1 | | |
| CK5 | Santa Cruz | + | 7 ± 1 | + | 3 ± 1 |
| CK18 | Santa Cruz | + | 5.5 ± 2.5 | + | 3.5 ± 0.5 |
| CK5/ | | + | 4.5 ± 0.5 | + | 2.5 ± 0.5 |
| CK18 | | | | | |
| p63 | Santa Cruz | + | 5 ± 2.5 | + | 7 ± 1 |
| AR | Santa Cruz | + | 14 ± 2 | ++/+++ | 5 ± 2 |
| CXCR4 | R&D | ++ | 10.5 ± 1 | +++ | 12 ± 2 |

*Mean percentage of cells expressing particular cell surface marker was calculated based on the three independent FACS analyses.
**PPT2 cells (unsorted before analysis, but established by previous repeated MACS-CD133⁺ cell sorting) were cultured on type I collagen-coated dishes in MSCB medium for 2, 4 and 8 weeks.
***Unsorted PC3MM2 cells were cultured on type I collagen-coated dishes in MSCB medium for 1-2 weeks.
+, ++ and +++ represents low, moderate and high expression.

### Example 2

### Establishment and Characterization of Highly Clonogenic PPT2 Cells

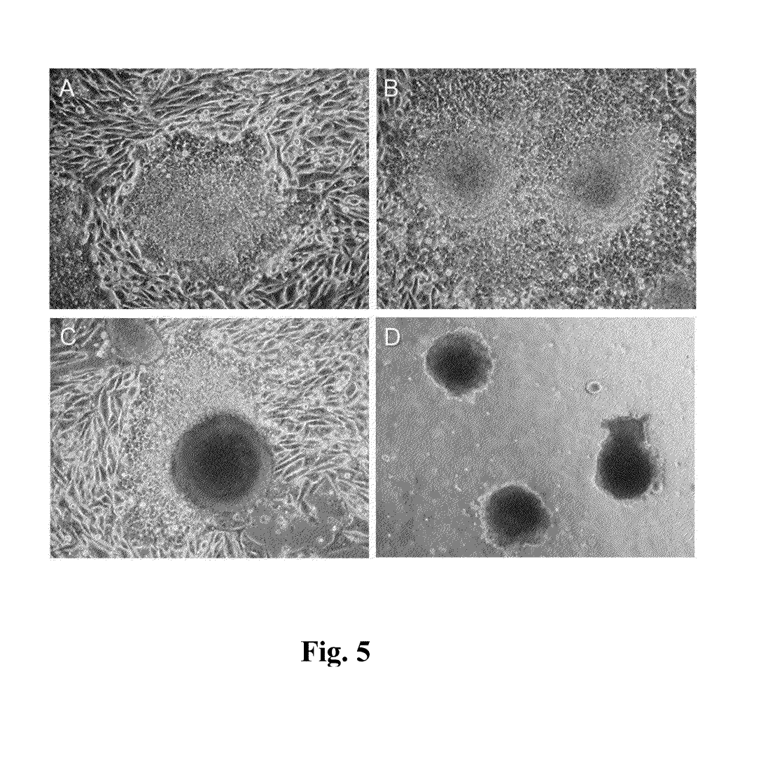

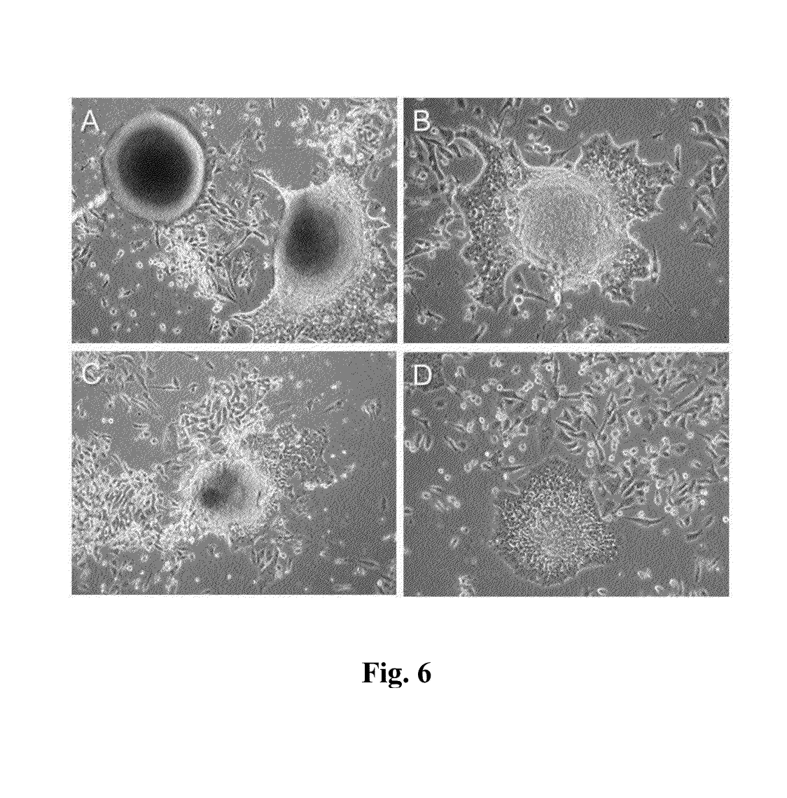

Approximately 8 months after the establishment of the parental PPT2 cell line, during which time multiple cell sortings and serial transplantation to the NOD/SCID mice were performed (as described in Example 1), a new subpopulation of small immature cells appeared as a round colonies (holoclones) surrounded by spindle-like much larger cells (FIG. 5A). The clonogenic and sphere-forming capacity of these cells is so high that the floating multicellular spheroids can be formed not only under non-adherent conditions, but also above the cells adherent to type I collagen (FIGS. 5B, C) and then detached (FIG. 5D). The pluripotent nature of the spheroid cells was confirmed by the following experiment: after plating of such spheroids on type I collagen-coated surfaces, new round colonies of small cells surrounded by larger spindle-like cells were formed (FIG. 6A-D).

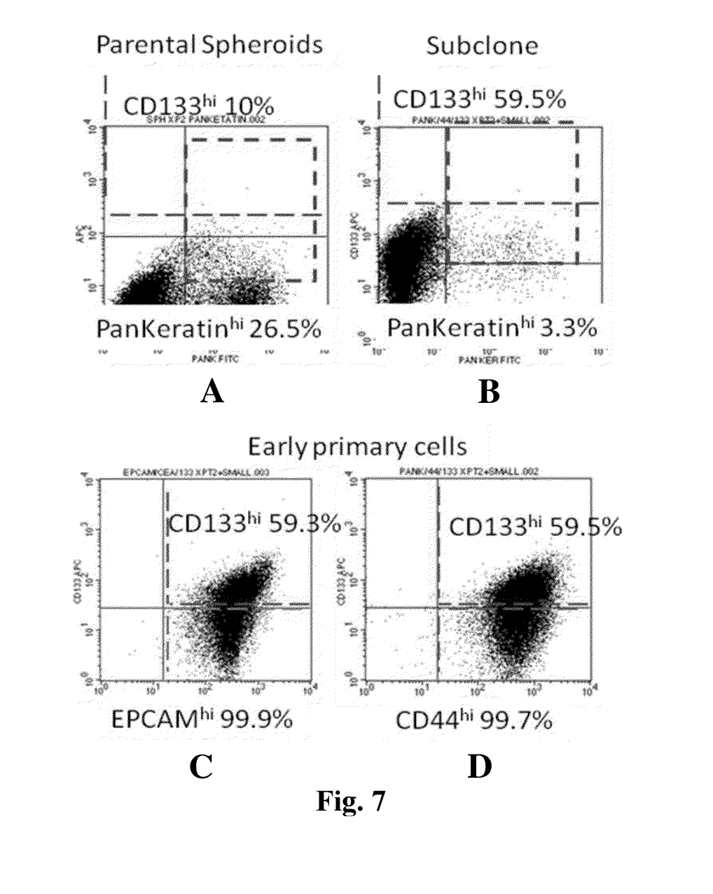

After clonal isolation, purification and propagation (as described in Example 1), these highly clonogenic cells of the PPT2 cell line, were subjected to FACS analysis to determine their phenotypic characteristics. The inventors found that 60% of the PPT2 cells expressed high levels of CD133 (CD133^{high}), and the entire population was positive for CD44 and CD326/epithelial cell adhesion molecule (EPCAM) (FIG. 7), all of which are common markers of stemness. In addition, only 3.3% of cells were differentiated (expressed PanKeratin kit; FIG. 7D), which reflects their immature, undifferentiated state characteristic for stem cells, in contrast to parental spheroid cells containing 26.5% differentiated cells. The PanKeratin kit is a cocktail of antibodies against different keratins (cytokeratins), which are intermediate filament proteins that are mainly expressed in epithelial cells. Keratin heterodimers composed of an acidic keratin (or type I keratin, keratins 9 to 23) and a basic keratin (or type II keratin, keratins 1 to 8) assemble to form filaments (Moll et al., 1982; Chang and Goldman, 2004). Keratin isoforms demonstrate tissue- and differentiation-specific profiles that make them useful as biomarkers (Moll et al., 1982).

After plating a known number of PPT2 cells on non-adherent ULA plates with serum-free MSCB media supplemented with 1% knock-out serum, clonogenic analysis revealed that approximately 30% to 50% of the PPSS cells were able to induce floating multicellular spheroids, which reflects the stem cell nature of these cells. These data are in line with the expression of the CD133^{high}, which additionally confirms the usefulness and feasibility of the CD133 cell surface marker for isolation of prostate CSCs. As has been previously determined (see, for example, Collins et al., 2005; Miki et al., 2007; Rowehl et al., 2008; Horst et al, 2008), purified CD133^{high}/CD44^{high} cells isolated from clinical specimens of metastatic PrC or highly metastatic cancer cell lines possess multiple stem cell characteristics and are highly tumorigenic and clonogenic.

These findings demonstrate that the PPT2 cell line represents an extremely rare, practically pure population of the primary prostate CSCs, which should be highly useful for the development of a novel generation of effective, CSC-targeted anti-cancer drugs. Also, this CSC line represent the unique model for basic and translational research focused on stem cell regulation and functioning, cancer development and progression, drug resistance and metastasis.

### REFERENCES CITED

Reya T, S. J. Morrison, M. F. Clarke, and I. L. Weissman, Stem cells, cancer, and cancer stem cells. Nature 414 (2001) 105-111.
Clarke M F, and M. Fuller, Stem cells and cancer: Two faces of eve. Cell 124 (2006) 1111-1115.
Collins A T, Berry P A, Hyde C, Stower M J, Maitland N J. Prospective identification of tumorigenic prostate cancer stem cells. Cancer Res, 65: 10946-10951, 2005.
Patrawala L, Calhoun T, Schneider-Broussard R, Li H, Bhatia B, Tang S et al. Highly purified CD44(+) prostate cancer cells from xenograft human tumors are enriched in tumorigenic and metastatic progenitor cells. Oncogene, 25: 1696-1708, 2006.
Miki J, Rhim J S. Prostate cell cultures as in vitro models for the study of normal stem cells and cancer stem cells. Prostate Cancer Prostatic Dis, 11(1):32-9, 2008.
Rowehl R H, Crawford H, Dufour A, Leyfman Y, Ju J, and Botchkina G I. Genomic Analysis of Prostate Cancer Stem Cells Isolated from Highly Metastatic Cell Line. Cancer Genomics and Proteomics. 5(6):301-309, 2008.
Klarmann G J, Hurt E M, Mathews L A, Zhang X, Duhagon M A, Mistree T, Thomas S B, Farrar W L. Invasive prostate cancer cells are tumor initiating cells that have a stem cell-like genomic signature. Clin Exp Metastasis, 26(5):433-46, 2009.
Bao S, Wu Q, McLendon R E, Hao Y, Shi Q, Hjelmelad A B, Dewhirst M W, Bigner D D and Rich A. Glioma stem cells promote radioresistance by preferential activation of the DNA damage response. Nature 444:756-760, 2006.
Dirks P B. Cancer: Stem cells and brain tumours. Nature 444, 687-688, 2006.
Eramo A, Ricci-Vitiani L, Zeuner A, Pallini R, Lotti F, Sette G, Pilozzi E, Larocca L M, Peschle C and De Maria R. Chemotherapy resistance of glioblastoma stem cells Cell Death and Differentiation 13, 1238-1241, 2006.
Todaro M, Alea M P, Stefano A B, Cammareri P, Vermeulen L, Iovino F, Tripodo C, Russo A, Gulotta G, Medema J P, Stassi GColon cancer stem cells dictate tumor growth and resist cell death by production of interleukin-4. Cell Stem Cell, 1:389-402, 2007.
Bleau A M, Hambardzumyan D, Ozawa T, Fomchenko E I, Huse J T, Brennan C W, Holland E C. PTEN/PI3K/Akt pathway regulates the side population phenotype and ABCG2 activity in glioma tumor stem-like cells. Cell Stem Cell. 4(3):226-35, 2009.
Tortoreto M, Fontanella E, Gatti L, Pratesi G, Fabbri A, Andriani F, Tinelli S, Roz E, Caserini R, Lo Vullo S, Camerini T, Mariani L, Delia D, Calabrò E, Pastorino U, Sozzi G. Highly tumorigenic lung cancer CD133+ cells display stem-like features and are spared by cisplatin treatment. Proc Natl Acad Sci USA, 106(38):16281-6, 2009.
Hutchinson E. Stem cells: Tumour stem cells generate vasculature. Nature Reviews Cancer 11, 4, 2011.
Shen M M and Abate-Shen C. Molecular genetics of prostate cancer: new prospects for old challenges. Genes Dev, 24(18):1967-2000, 2010.
Mimeault M, Batra S K. New advances on critical implications of tumor- and metastasis-initiating cells in cancer progression, treatment resistance and disease recurrence. Histol Histopathol, 25(8):1057-73, 2010.
Friedrich J, Siedel C, Ebner R, Kunz-Schughart L A. Spheroid-based drug screening: considerations and practical approach. Nature Protocols 4:309-324, 2009.
Kirkland S C. Type I collagen inhibits differentiation and promotes a stem cell-like phenotype in human colorectal carcinoma cells. Br J. Cancer. 101(2):320-326, 2009.
Miki J, Furusato B, Li H, Gu Y, Takahashi H, Egawa S et al. Identification of putative stem cell markers, CD133 and CXCR4, in hTERT-immortalized primary nonmalignant and malignant tumor-derived human prostate epithelial cell lines and in prostate cancer specimens. Cancer Res; 67: 3153-3161, 2007.
Horst D, Kriegl L, Engel J, Kirchner T, Jung A. CD133 expression is an independent prognostic marker for low survival in colorectal cancer. Br J Cancer, 99(8):1285-9, 2008.
Moll R, Franke W W, Schiller D L, Geiger B, Krepler R. The catalog of human cytokeratins: patterns of expression in normal epithelia, tumors and cultured cells. Cell. 1982 November; 31(1):11-24.
L Chang, R D Goldman. Intermediate filaments mediate cytoskeletal crosstalk. Nat Rev Mol Cell Biol, 5 (2004), pp. 601-613

1. A cancer cell line comprising: at least 2% CD44+ cells; at least 2% CD133+ cells; cells capable of anchorage-independent growth in culture; and cells capable of forming tumors in a xenograft model.

2. The cancer cell line of claim 1, comprising at least 50% CD44+; CD326/epithelial cell adhesion molecule (EPCAM)+ cells and at least 20% CD133+; CD44+; EPCAM+ cells.

3. The cancer cell line of claim 2, wherein the cell line is a prostate cancer cell line.

4. The cancer cell line of claim 3, wherein said cell line is the PPT2 cell line.

5. A method of creating a cell line enriched for cancer stein cells, the method comprising: isolating tumor-derived cells that show fast adherence in culture; culturing fast adherent cells under stem cell-promoting conditions; and performing one or more rounds of cell sorting to enrich the population of cells expressing stem cell markers.

6. The method of claim 5, wherein the stem cell-promoting conditions include one or more of: culturing cells in serum-free media; culturing cells on low-attachment culture surfaces; serial passage for two or more rounds on low-adherent culture surfaces; and treatment under cytotoxic conditions.

7. The method of claim 5, further comprising the step of performing at least one transplantation of cells in a xenograft model animal, followed by further culturing of cells from the xenograft-derived tumor.

8. The method of claim 5, further comprising the steps of isolation and culturing of spheroid cells which show attachment-independent growth in culture.

9. The method of claim 5 wherein the cancer is prostate cancer.

10. A method of identifying a candidate compound with antiproliferative activity, the method comprising: contacting a cancer cell from the cell line of claim 1 with the selected candidate compound; monitoring proliferation of the cancer cell; and identifying the candidate compound as an antiproliferative agent if the candidate compound inhibits proliferation of the cancer cell relative to proliferation of a cancer cell of the same cell type that is not contacted with the candidate compound.

11. A pharmaceutical composition comprising a candidate compound identified by the method of claim 10.

12. A pharmaceutical composition comprising a derivative of a candidate compound identified by the method of claim 10.

13. The cancer cell line of claim 1, wherein the cell line is a prostate cancer cell line.

14. The method of claim 10, wherein the cancer cell is from the cancer cell line of claim 2.

15. The method of claim 10, wherein the cancer cell is from the cancer cell line of claim 3.

16. The method of claim 10, wherein the cancer cell is from the cancer cell line of claim 4.

17. A pharmaceutical composition comprising a candidate compound identified by the method of claim 14, or a derivative thereof.

18. A pharmaceutical composition comprising a candidate compound identified by the method of claim 15, or a derivative thereof.

19. A pharmaceutical composition comprising a candidate compound identified by the method of claim 16, or a derivative thereof.
